(19) United States
(12) Patent Application Publication
(10) Pub. No.: US 2015/0245911 A1
(43) Pub. Date: Sep. 3, 2015

(54) REINFORCED MULTI-LAYERED MEMBRANE

(71) Applicant: Shih-Liang Stanley Yang, Laguna Hills, CA (US)

(72) Inventor: Shih-Liang Stanley Yang, Laguna Hills, CA (US)

(21) Appl. No.: 14/635,940

(22) Filed: Mar. 2, 2015

Related U.S. Application Data
(63) Continuation-in-part of application No. 14/194,722, filed on Mar. 1, 2014.

(51) Int. Cl. A61F 2/28; A61L 27/16

(52) U.S. Cl. CPC A61F 2/2846; A61L 27/16

(57) ABSTRACT

A multi-layered membrane may include a first layer made by polytetrafluoroethylene polymer (PTFE), a second layer made by fluorinated ethylene propylene polymer (FEP), a third layer made by a reinforcing unit, and a fourth layer made by PTFE. The second and third layers are sandwiched between the first and the fourth layers, and the reinforcing unit is configured to span a bone cavity and used for fixation to bone surrounding the bone cavity. The reinforcing unit is configured to form a desired shape prior to placement on the bone defect and maintain the formed shape upon placement. In one embodiment, the reinforcing unit has multiple elongated members forming a junction or junctions, and the elongated members have a free distal end that extends away from the junction; and at least one elongated member has an enlarged appendage formed in the free distal end spaced apart from the junction.

### CROSS-REFERENCE TO RELATED APPLICATIONS

This application is a continuous-in-part (CIP) application of U.S. Ser. No. 14/194,722, filed on Mar. 1, 2014, which claims priority under 35 U.S.C. 119 (e) to U.S. Provisional Patent Application Ser. No. 61/771,605, filed on Mar. 1, 2013. The entire contents of which are incorporated herein by reference.

### FIELD OF THE INVENTION

The present invention relates to implantable biomaterials, and more particularly to reinforced multi-layered membranes.

### BACKGROUND OF THE INVENTION

Polytetrafluoroethylene (PTFE) and expanded polytetrafluoroethylene (ePTFE) materials are commonly used biocompatible materials. Articles made by PTFE and ePTFE are used in many medical applications. For example, PTFE and ePTFE membranes are used in guided tissue regeneration or guided bone regeneration for bone grafting applications. Guided bone regeneration (GBR) and guided tissue regeneration (GTR) are dental surgical procedures that utilize barrier membranes to direct the growth of new bone and gingival tissue at sites having insufficient volumes or dimensions of bone or gingival for proper function, esthetics or prosthetic restoration.

Recently, it has been discovered that the use of a flexible high-density polytetrafluoroethylene (PTFE) sheet material is useful in guided tissue regeneration. High density PTFE is substantially nonporous, so it would not incorporate with cells or attach to fibrous adhesions. By presenting a smooth surface to the biological materials, a high density PTFE barrier is easily inserted and removed following extended implantation periods. An example of a high density PTFE barrier material is disclosed in U.S. Pat. No. 5,480,711. While high density PTFE medical barriers provide advantages over macro porous barriers, the smooth surface of the high density PTFE barriers sometimes leads to dehiscence of the soft tissue overlying the barrier. The dehiscence problem is caused by the fact that the smooth surface of high density PTFE will not incorporate cells and will not attach to fibrous adhesions. Thus, over the course of healing, the incision will occasionally split open over the high density PTFE barrier.

Moreover, while PTFE and ePTFE membranes have been used frequently in medical applications, current design of PTFE and ePTFE membranes may be disadvantageous due to shrinkage during implantation. Even though the mechanism of the shrinkage is not known or reported so far, it is suspected that some PTFE and ePTFE membranes may be under tension due to the expansion and/or calendaring process imposed during the process of manufacturing the membranes. Over a long period time of implantation, for example over six months immersed in the body fluid, the ePTFE membrane under tension may relax and shrink.

U.S. 2011/0060420 to BARTEE et al. discloses a composite multi-layered material may generally comprise a d-PTFE material combined with an open structured material (either resorbable or non-resorbable) creating a composite multi-layer material. Attachment of the layers may be accomplished by stitching layers of material, exertion of hydraulic or other pressure, application of a biocompatible adhesive or heat, or some combination of the foregoing. However, BARTEE did not disclose a composite multi-layer material with a reinforced unit that can significantly enhance the structure of the composite material for bone grafting applications. Upon introducing the reinforced unit in the multi-layered material, delamination may have occurred easily.

Fluorinated ethylene propylene (FEP) has been widely used as an adhesive in the areas of biomaterials and biocompatible implant devices. U.S. Pat. No. 6,159,565 to Campbell et al. discloses an intraluminal vascular graft in the form of a tube of porous expanded polytetrafluoroethylene (PTFE) wherein the porous PTFE film has a microstructure containing a multiplicity of fibrils oriented substantially parallel to each other. The tube has a wall thickness of less than about 0.25 mm and is made from at least one first layer and at least one second layer of porous PTFE film. In a preferred embodiment, an adhesive such as FEP can be used between the PTFE films. Like BARTEE discussed above, Campbell did not disclose a multiple-layered structure containing a reinforced unit for bone grafting applications. Upon introducing the reinforced unit in the multi-layered material, delamination may have occurred easily. U.S. Pat. No. 8,197,529 to Cully also discloses coating FEP on an ePTFE stent graft.

GTR or GBR membranes made by PTFE and ePTFE reinforced with a titanium binder such as those disclosed in U.S. Pat. No. 8,556,990 (“the '990 patent”) tend to delaminate during handling prior to administration. Delamination of the titanium reinforced PTFE or ePTFE membranes may result in unusable products. If delamination occurs after the product has been implanted, it may lead to surgical complications, potentially leading to failure of bone grafting procedures. While the mechanism of membranes delamination has not been reported, it is suspected that the delamination may have occurred due to poor bonding strength between the layers of PTFE membranes, ePTFE membranes, or PTFE and ePTFE membranes, and between the membranes and the titanium reinforcing elements. Although the '990 patent vaguely mentioned about the use of adhesive, it does not disclose if what adhesive could be used and under what conditions, and whether the use of such adhesives would improve the bonding strength and minimize the occurrence of delamination to any significant degree.

Furthermore, in the '990 patent, the reinforcing titanium binder embedded in the multi-layered PTFE membranes is bendable and may have one or more slender elongated members such that the titanium binder can be formed to obtain a desired shape, or can be adjusted to obtain the desired shape prior placing on the bone defect and maintain the desired shape upon placement. However, the multi-layered membrane with the titanium binder therein disclosed in the '990 patent may be difficult to apply on the bone defect during dental surgical procedures.

Regarding the reinforcing titanium binder in the '990 patent, it has multiple elongated members forming a junction, and the elongated members have a free distal end that extends away from the junction, wherein at least one of the elongated member has a predrilled hole formed in the free distal end spaced apart from the junction. The predrilled hole is suitable for receiving a fastener that passes through the PTFE membrane and into an area of bone. A fastener such as a surgical pin or screw is used to secure the multi-layered reinforced material to the surgical site.

Depending upon the size and configuration of the bony defect and the dimension of the multi-layer membrane utilized to cover the defect, up to 7 fasteners, including, two fasteners crestally, three fasteners buccally and two fasteners lingually, may be used to secure a multi-layered reinforced material to the surgical site. Proper fixation of the membrane to the adjacent bone and close conformance of the membrane to the packed bony defect are required to prevent the movement and migration of underlying bone grafting material during bone regeneration. However, handling and applying tiny surgical fasteners of about 5 mm in length and 1.5 mm in diameters in the predrilled hole of a slender elongated member as disclosed by U.S. Pat. No. 8,556,990, to secure a multi-layered membrane in a tight oral environment is very challenging and require the use of a custom designed bone screw fixation kit. Also, some fixation points may not be in the direct line of sight. The complex and difficult procedure required to use custom made bone screw kit in a tight oral space at multiple fixation points often discourages practitioners to use the multi-layered membranes. The custom made bone screws or bone pins are usually expensive, and tend to drop from the bone screw fixation kit during the fastening procedures. If the bone screw is dropped in the oral chamber, it needs to be removed or the patient may accidentally swallow the fastener. Therefore, there remains a need for a new and improved membrane with an improved design of the reinforcing binder that reduce the amount of multiple fasteners needed to secure the membrane and/or facilitate the insertion of a surgical fastener will greatly reduce the time and stress of practitioners to administer the membrane and lessen the pain and suffering induced on the patient.

### SUMMARY OF THE INVENTION

The present invention provides a medical barrier that includes a sheet of micro porous, expanded polytetrafluoroethylene (ePTFE) polymer material having a density in a range of about 0.3 gm/cc to about 1.2 gm/cc, and preferably in the range of about 0.4 gm/cc to about 1.0 gm/cc. Preferably, the sheet has at least one textured surface, and has substantially the needed strength required for the applications in all directions. The sheet of medical barrier of the present invention has a thickness in a range of about 0.01 mm to about 1.00 mm, preferably in the range of about 0.05 mm to about 0.50 mm, and more preferably in the range of about 0.1 mm to about 0.3 mm. The textured surface may include a continuous dotted pattern of holes, and preferably a continuous woven pattern or a continuous pattern of hills and valley formed in the surface of the sheet. If the sheet has only one textured surface, the valley or the indentations have a depth less than the thickness of the sheet and each valley or indentation has a width of less than 0.5 mm, and preferably less than 0.3 mm. The textured patterns are distributed substantially uniform over the surface of the sheet. If the sheet has textured pattern on both surfaces, the valley or the indentations have a depth less than half of the thickness of the sheet. The textured pattern is repeated at less than 500 microns, preferably less than 200 microns and more preferably at less than 100 microns are distributed uniformly over the surface of the sheet.

In addition, the sheet of the ePTFE medical barrier of the present invention is micro porous and has a porosity selected from the following ranges: (A) from about 5% to about 20%, (B) from about 20% to about 40%, (C) from about 40% to about 60%, and (D) more than 60%. Preferably, the ePTFE membrane has a density of less than 1 g/cc, and has an average fibril length selected from the following ranges: (A) less than 60 microns, (B) less than 30 microns, (C) less than 15 microns, and (D) less than 10 microns.

Fluorinated ethylene propylene (FEP) is a copolymer of hexafluoropropylene and tetrafluoroethylene. It differs from PTFE resins in that it is melt-processible using conventional injection molding and screw extrusion techniques. It is noted that FEP has a melting point of 260° C. and PTFE decomposes before melting. The melting point of FEP is significantly lower than that of the decomposition temperature of PTFE. ePTFE is made by PTFE in an expansion process, so the thermal and chemical properties are substantially similar to PTFE. Different grades of FEP films ranging from 1 μm in thickness and up are commercially available. Alternatively, the FEP film may be introduced by sprinkling or spraying of FEP micro powders or by spraying a solution of FEP dispersions onto the PTFE or ePTFE membranes before subjecting the layered films under heat and pressure. FEP dispersions are colloid solution containing FEP micro particles suspended in water. The micro particles dispersed onto the PTFE and ePTFE membranes form a FEP film under heat and pressure to bond to the ePTFE membrane.

Through experiments, we found that it is most effective to produce the biomaterial according to the present invention when the thickness of the FEP film is preferably less than or equal to 2 μm, more preferably less than or equal to 1 μm, and still more preferably less than or equal to 0.5 μm. If FEP dispersion is used, it should render an FEP thin film of about less than or equal to 0.5 μm. If the FEP film is too thick, the resulting multi-layered biomaterial may not have sufficient flexibility for intended applications. Regardless of various configuration design for intended medical applications, the thickest cross section of the multi-layered biomaterial according to the present invention should preferably be less than or equal to 2 millimeters, more preferably be less than or equal to 1 millimeter, still more preferably be less than or equal to 0.5 millimeters, and still more preferably less than 0.25 mm If the FEP film is too thick, the resulting multi-layered biomaterial may not have sufficient flexibility to satisfy the implant applications. Preferably, the multi-layered biomaterial should have a thickness of less than or equal to 0.25 millimeter and should have an initial (before bonding) FEP thickness of less than or equal to 0.5 μm.

Biocompatible membranes of the present invention can be made by laminating an FEP film to a layer of PTFE or ePTFE membranes, or between two layers of PTFE membranes, ePTFE membranes, or PTFE and ePTFE membranes under heat and pressure. After lamination, the multi-layered composite material is found to have excellent biocompatible properties, comparing with the membrances disclosed in the prior arts. In particular, the composite material is found to be more resistant to delamination and shrinkage than the material disclosed in the prior arts. Moreover, it is still flexible, conformable and biocompatible for the intended GTR and GBR procedures.

Furthermore, multi-layered membranes embedded with certain configurations of reinforcing titanium binder exhibit better conforming properties than prior arts. In fact, the use of such multi-layered membrane may need less fixation points because of its improved conforming properties to the underlying adjacent bone. In one embodiment, the titanium binder has multiple elongated members forming one or more junctions, and the elongated members have a free distal end that extends away from the junction, wherein at least one of the elongated member has an enlarged appendage formed in the free distal end spaced apart from the junction. The enlarged appendage portion of the elongated membrane substantially differs from the slender elongated members by its configuration, including size, shape and/or thickness. The location, configuration and size of the appendage are selected to improve the conforming attachment of the multi-layered membranes to the adjacent bone around the cavity, but do not negatively affect the overall drapability of the membranes, including, bendability, twistability, stretchability, space maintenance, and overall utility of the multi-layered membranes.

The reinforcing binder having an appendage located at the free distal end of the joint elongated members can be made of other materials in addition to titanium or surgical grade titanium. For example, the reinforcing titanium binder can be made of any material suitable for medical applications, including implant application that exhibit suitable bendability, compatibility with the enclosing membranes and have a young's modulus or stiffness higher than the enclosing membranes to reinforce the conformance properties of the resulting multi-layered membranes. Such material may be derived from a variety of materials, including polymers, metallic materials, ceramic material, fibrous material or natural polymers, including human or animal tissue, such as collagen, or a composite of the above materials.

The reinforced multi-layered PTFE materials made according to the present invention can be used for medical applications, particularly for implant applications, where maintaining consistent property and material integrity over a long period of time is very important. In addition, such material should be able to withstand rigorous biocompatibility tests, including in vitro and in vivo tests, as stipulated in ISO 10993 series of standard biocompatibility tests, wherin ISO stands for International Organization for Standardization, and should be able to withstand commercial sterilization processes, such as ethylene oxide gas sterilization, or autoclave without siginificantly affecting the property of the material. Moreover, the sterilized material should have at least two years, and at best three years of shelf life to satisfy logistical and distribution needs. In addition, the biomaterials as prepared according to the present invention should be able to withstand the process of administration, and long term implantation without delamination or significant shrinkage. The biomaterial in the present invention can be made in various configurations as barrier membranes for treating periodontal disease and defects, and for use as a hernia mesh to reconstruct soft tissue defects of the chest and abdomen.

### BRIEF DESCRIPTION OF THE DRAWINGS

FIG. 1 illustrates a schematic view of one embodiment in the present invention.

FIG. 2 illustrates a schematic view of another embodiment in the present invention.

FIG. 3 illustrates a schematic view of the FEP layer with a plurality of holes in the present invention.

FIGS. 4A to 4E illustrate schematic views of different designs of appendages in the present invention.

### DETAILED DESCRIPTION OF THE INVENTION

The detailed description set forth below is intended as a description of the presently exemplary device provided in accordance with aspects of the present invention and is not intended to represent the only forms in which the present invention may be prepared or utilized. It is to be understood, rather, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described can be used in the practice or testing of the invention, the exemplary methods, devices and materials are now described.

All publications mentioned are incorporated by reference for the purpose of describing and disclosing, for example, the designs and methodologies that are described in the publications that might be used in connection with the presently described invention. The publications listed or discussed above, below and throughout the text are provided solely for their disclosure prior to the filing date of the present application. Nothing herein is to be construed as an admission that the inventors are not entitled to antedate such disclosure by virtue of prior invention.

As discussed above, delamination of the PTFE or ePTFE membranes may lead to surgical complications, and more seriously failure of bond grafting procedure. To solve these problems, many experiments have been conducted to improve the bonding strength of the multi-layer PTFE membrances without significantly affecting the dimension, biocompatibility, pliability, malleability and the conformability of the membrane for bone grafting applications. To achieve this goal, it was found that introducing one or more adhesive layers in between the PTFE membranes will significantly improve the bonding strength without significantly impacting the general properties of the membranes for bone grafting applications. A suitable adhesive should meet the following criteria:

Thickness: the thickness of the adhesive layer has to be less than 0.002″, preferably 0.001″, and more preferably less than 0.005″. If the adhesive is too thick, it may affect the overall thickness and malleability of the membranes.

Thermal Property: the adhesive's melting point should be substantially below the melting point or decomposition point of the PTFE, such that the melting of the adhesive layer will not affect the strength and dimension of the PTFE layers. If the adhesive's melting point is too high and too close to the melting or decomposition point of the PTFE membranes, it may affect the property of the PTFE membranes.

Chemical property: the adhesive layer should be substantially hydrophobic and compatible with PTFE membranes. The hydrophobicity of the materials is typically determined by the use of (i) water contact angle by goniometry, and (ii) critical surface tension by the Zisman method or by the wetting tension method. Preferably the contact angle of the adhesive should be greater than 95 degree, more preferably greater than 100 degree and still more preferably greater than 105 degree. The critical surface tension of the adhesive should be less than 32 dynes/cm, preferably less than 25 dynes/cm, and still more preferably less than 20 dynes/cm. If the adhesive materials do not have similar chemical properties they will not be able to fuse with the PTFE membranes and bond the PTFE layers together.

Space maintenance: The incorporation of the adhesive layer should not affect the overall utilities, including biocompatibility and the space maintenance ability of the titanium reinforced multi layer membrane.

Processing Conditions: the adhesive may be sprayed onto the PTFE layer or apply as one or more layers within the PTFE layers. In one embodiment, one adhesive layer is lay on a sheet of PTFE membrane, the Titanium binder is lay on the adhesive layer, and another sheet of PTFE membrane is lay on top of the multi-layers. The processing conditions mainly involving the control of temperature and pressure should effectively fuse the adhesive layer into the adjacent surface of PTFE layers, and upon cooling and the release of pressure should results in the bonding of the multi-layer components. The optimal processing conditions depend on the kind of adhesive used and the kind of the PTFE membrane utilized. In general, effective conditions should result in the melting of the adhesive and fusing of the molten adhesive molecules into the crevices of PTFE membranes, or result in the blending of the molten adhesive molecules with the superficial molecules of the PTFE membranes. Thus, upon cooling and return to the ambient condition, the multi-layers components are physically bonded and strengthened.

The present invention provides a method of repairing a defect in alveolar bone underlying gingival tissue, which comprises steps of: (a) placing a sheet of textured, micro porous ePTFE material having a density in a range of about 1.0 gm/cc to about 0.3 gm/cc, over said defect between the bone and the gingival tissue with said textured surface in contact with said gingival tissue; (b) securing the gingival tissue over the sheet, allowing the defect to heal under the sheet; and (c) removing the sheet after the defect has healed.

The micro porous ePTFE membrane may be secured in a place by the use of a biocompatible adhesive, and preferably by the use of sutures or filaments. The density of ePTFE is the ratio of the mass of a given sample of expanded PTFE to its volume. It determines the amount of void space and the microstructures of the material which allows nutrient diffusion across the membrane material and enhances tissue attachment to the micro porous surface.

Furthermore, ePTFE is an inert and biocompatible material with a history of medical implant use. U.S. Pat. Nos. 3,953,566, 4,187,390, 6,702,971 and 7,374,679 (the disclosures of which are incorporated herein by reference) teach methods for producing ePTFE and characterize its porous structure. The microstructure of ePTFE is a three-dimensional matrix of nodes connected by fibrils. The pore size of ePTFE can be characterized by determining the bubble point and the mean flow pressure of the material. Bubble point and mean flow pressure are measured according to the American Society for Testing and Materials Standard ASTM F316-03(2011) using alcoholic solution.

The fibril length of ePTFE is defined herein as the average length of fibrils between nodes connected by fibrils in the direction of expansion. PTFE expanded in one or more than one direction are thought to be equally applicable to the invention. The measurement of average fibril length and pore sizes are well known to those skilled in the art and are disclosed in references cited herein, including U.S. Pat. No. 5,032,445, and in ASTM F316-03(2011).

It is known to those skilled in the art that the microstructures of ePTFE depended on the processing conditions of the manufacturing process, in particularly the stretching ratio, the stretching rate and the amount of volatile components contained within the material. It is also known that there is no single parameter that can precisely characterize the microstructures of the ePTFE material due to the complex geometry of the node and fibril microstructures. Some lower density ePTFE, thus higher porosity, may have shorter average fibril length than higher density ePTFE, and vice versa. The measurement of average fibril length is well described in U.S. Pat. No. 5,032,445. The node and fibril structures could be present in a tortuous path fashion rather than straight vertically across the membrane. This also complicates the characterization of microstructures of micro porous ePTFE. In addition, certain processes produce asymmetrical ePTFE sheets, meaning the porosity of the surface layers is different from the bulk or the central portion of the ePTFE.

U.S. Pat. No. 5,032,445 teaches the use of macro porous ePTFE material with average fibril lengths greater than about 60 microns, preferably greater than about 100 microns, ethanol bubble points of less than about 2.0 psi, preferably less than about 0.75 psi, ethanol mean flow pressure less than about 10 psi, preferably less than about 3.0 psi, and densities less than about 1 g/cc and preferably about 0.3 to about 0.1 g/cc to enhance connective tissue ingrowth for use in guided tissue regeneration in the repair of bone defects. During wound hearing, tissue grows into and integrates with the porous material. While the tissue incorporation into the material stabilizes the wound site, it presents a difficult problem to the patient and the surgeon during the removal process. The incorporated cells and fibrous connective material may make removal of the barrier painful and traumatic to the patient, and it is very time consuming and difficult for the surgeon. In addition, the use of macro porous biomaterials in the oral cavity may result in early bacterial contamination of the material. Bacterial contamination of macro porous biomaterials may result in antibiotic-resistant infection, which can require early removal of the device.

U.S. Pat. No. 5,957,690 teaches the use of an unsintered, high density PTFE membrane with discrete, plural indentations of about 0.5 mm in width distributed uniformly throughout the surface of the material, and a density ranging from 1.2 g/cc to about 2.3 g/cc. Such unsintered, high density PTFE materials are essentially non-porous, and are featureless when examined by the use of scanning electron microscope even at micron level. While these materials successfully block off the ingrowth of tissue and contamination of bacteria, the risk of dehiscence and splitting of the gingival tissue overlying the membrane still exists due to limited surface attachment and poor diffusion of nutrients across the non-porous membranes. The discrete superficial macro indentations exhibit limited improvement in enhancing surface attachment. This invention provides a sheet of micro porous ePTFE membrane that exhibits micro surface texture, which is characterized by three-dimensional node and fibril microstructure as illustrated by U.S. Pat. No. 4,187,390, at a level relevant to and discernible by the creeping cells and tissue, but limits the deep penetration of bacteria and ingrowth of tissue into and across the membrane material. The micro porous ePTFE of the present invention allows nutrients to diffuse across the membrane to maintain the health of the thin tissue overlying the membrane, and in addition, to provide multi-fold increase in surface areas for tissue and cells to anchor and attach onto micro porous surface comparing with non-porous high density PTFE and macro porous ePTFE membranes. Bacteria colonization is blocked off by the combination of the small pore size and the low surface tension of the ePTFE material or compartmentalized in the highly hydrophobic micro pores and cannot multiply. The upper boundary of micro porous structures is limited by the deep ingrowths and integration of tissue within the material that will result in lengthy and traumatic removal of the ePTFE material, and/or the colonization of bacteria within the micro porous material that will cause infection and/or inflammation. The lower threshold of the micro porous structures is limited by the adequate diffusion of nutrients across the membranes to support the healthy growth and metabolism of the overlying tissue and cells.

The diffusion of nutrients across the membranes, such as glucose or body fluids, may be determined using diffusion chambers, wherein one chamber filled with nutrients dissolved in a simulated body fluid solution is separated by a barrier membrane from a neighboring chamber containing only simulated body fluid solution. The rate of diffusion of the nutrients across the membrane, if any, is determined by measuring the concentration of the nutrients diffusing into the neighboring chamber at different time durations. Bacteria penetration across the membrane, if any, may be determined using a similar method.

Preferably, the micro pores of ePTFE membrane will facilitate the tissue to adhere and attach to the surface without ingrowth deeper than one, two, to several cellular length, and support the healthy growth and metabolism of the tissue while the bone is regenerated under the membrane. More importantly, the micro pores of ePTFE membrane would develop biofilm on the surface of the ePTFE membrane and prevent bacteria from colonizing or penetrating across the barrier membrane to cause inflammation and/or infection. Also, the membrane will support the growth and attachment of the tissue without inflammation and/or infection caused by bacteria contamination or colonization, and yet still removable by non-surgical removal procedures.

Non-surgical removal procedures are defined as non-traumatic, and retrievable by the use of a forceps to grasp the membrane and pull off gently from the wound site after a small incision (for primary closed situation), or non-traumatic, and retrieved by the use of a curette to separate the adhered tissue from the material, then removed by the use of a forceps. For a non-primary closed case, no incision is needed as a part of the membrane is exposed throughout the wound healing, and the membrane can be removed using the procedures described above without an incision.

Preferably, the average superficial fibril length of the micro porous ePTFE membrane of the present invention is selected from one of the following ranges: (A) from about 40 micron to 60 microns, (B) from about 30 microns to 40 microns, (C) from about 20 microns to 30 microns, (D) from about 10 microns to 20 microns, and (E) from about 0.1 microns to 15 microns. The density of the micro porous membrane is preferably selected from one of the following ranges: (A) from about 0.3 g/cc to 1.2 g/cc, (B) from about 0.3 g/cc to about 0.5 g/cc, (C) from about 0.5 g/cc to about 0.8 g/cc) and (D) from about 0.8 g/cc to about 1.1 g/cc. The bubble point pressure measured on the micro porous ePTFE membrane of the present invention is selected from one of the following ranges: (A) greater than 1.5 psi, (B) greater than 2.0 psi and (C) greater than 3.0 psi.

Depending upon clinical applications and requirements, the sheet of medical barrier of the present invention may have a thickness in a range of about 0.1 mm to about 3 mm, preferably in the range of about 0.10 mm to about 1.00 mm, and more preferably in the range of about 0.1 mm to about 0.3 mm. The textured surface comprises a continuous dotted pattern of specific features, including holes, and preferably a continuous woven pattern, a continuous mesh-like pattern, or a continuous pattern of hills and valleys formed on the surface of the sheet. If the sheet has only one textured surface, the valley or the indentations have a depth less than the thickness of the sheet and each valley or indentation has a width of less than 1.0 mm, and preferably less than 0.5 mm. The textured patterns are distributed substantially uniform over the surface of the sheet. If the sheet has textured pattern on both surfaces, the valley or the indentations have a depth less than the half the thickness of the sheet. The textured pattern is preferably repeated at less than 500 microns, preferably less than 200 microns and more preferably at less than 100 microns distributed uniformly over the surface of the sheet. Such woven, hills-and-valley or grooves patterns provides more surface areas at the macro level and are more conducive for tissue to anchor and attach than the pattern consisting of plurality of discrete indentations taught by the prior arts.

The barrier of the present invention is made by first forming a thin sheet of micro porous ePTFE and then embossing the sheet with a metal or plastic mesh. Manufacturing of ePTFE is well known to those skilled in the art. U.S. Pat. Nos. 3,953,566, 4,187,390, 6,7029,71 and 7,374,679 (the disclosures of which are incorporated herein by reference) teach methods for producing ePTFE and characterize its porous structure. An appropriate process is selected to make thin flat sheets of the desired thickness, a desired density and desired micro porous structures, including, average fibril length and bubble point pressure, and having substantially uniform strength in all directions. The resulting flat sheet has two substantially smooth surfaces. After the sheet is made and trimmed to the appropriate size, it is embossed to form a desired texture in one or both of its surfaces. In the preferred embodiment, the embossing step is performed by placing a sheet of patterned metal or polymer mesh on top of the unembossed sheet of ePTFE. The patterned metal or polymer sheet material, such as polyethylene or polypropylene, is harder and has more compressive strength than the micro porous ePTFE material. One of the preferred mesh is a fine pore-size titanium mesh manufactured by Unicare Biomedical, Inc. (California). The titanium mesh has a pattern that is embossed into the polymer sheet. The titanium mesh and the ePTFE sheet are passed together between a pair of rollers, which emboss the pattern of the titanium mesh into one or both surface of the ePTFE sheet. After embossing, the embossed ePTFE sheet may be cut into smaller sheets of various shape and size for packaging and distribution.

From the foregoing, it may be seen that the medical barrier of the present invention overcomes the shortcomings of the prior arts. In one embodiment, the present invention provides a micro porous ePTFE sheet that allows the attachment and ingrowth of cells and tissue onto the ePTFE sheet or into the ePTFE sheet within one, two, to several cell length in depth, but not across the sheet material, and the tissue can still be separated from the barrier membranes by gently pulling or peeling apart from the micro porous sheet with non-surgical and non-traumatic procedures. In another embodiment, the present invention due to the presence of three-dimensional node and fibril structure at the micron level provides significantly more surface areas for attachment and anchoring to facilitate tissue attachment than conventional high density PTFE material and macro porous material. In still another embodiment, the present invention provides an ePTFE barrier membrane that exhibits surface texture both at the micro level with three-dimensional node and fibril structure, and at the macro level created by the embossing process, which is not envisioned by the prior arts. In still another embodiment, the present invention provides an ePTFE sheet with surface textures that can be tailored at the micro level by adjusting the micro porosity of the barrier and at the macro level by controlling the embossing process. Such flexibility and advantages accompanied with the features are not envisioned and disclosed by the prior arts.

Such micro porous ePTFE that facilitates cell and tissue attachment, but limits its penetration to one, two or just several cellular layer is particularly well adapted for use in medical applications that requires non-traumatic, non-surgical removal of the ePTFE membranes after wound healing or tissue regeneration is completed. This medical barrier of the present invention is particularly useful in guided tissue regeneration in the repair of bone defects, such as in the repair of alveolar bone defects. The barrier prevents rapidly migrating gingival tissue cells from entering the defect and allows the alveolar bone to regenerate. At the same time, the barrier allows the nutrients to diffuse through the barrier to maintain the healthy attachment, growth and metabolism of the gingival tissue. During healing, the gingival tissue adheres to the textured surface of the barrier to anchor the gingival tissue over the barrier, thereby preventing dehiscence or splitting open of the tissue covering the material. However, the pore sizes are limited to an extent that it prevents the gingival tissue from growing into and integrate with the barrier. Thus, after the bone defect has healed, the barrier may be removed with a minimum of trauma to the gingival tissue.

### EXAMPLE 1

### Making Textured ePTFE Membrane

A sheet of micro porous expanded PTFE membrane having a density of 0.8 g/cc, a thickness of 0.3 mm and an average fibril length of 3 microns made according to the prior arts disclosed above are used for the study. The ePTFE membranes were trimmed into appropriate width and sandwiched between two sheets of thin titanium mesh (Cytoflex® Mesh, by Unicare Biomedical, Inc.). The titanium mesh has a thickness of 0.004″, a hole diameter of 0.010″ and a hole edge-to-edge distance of 0.005″. The titanium mesh and the ePTFE sheet are passed together between a pair of rollers, which emboss the pattern of the titanium mesh into both surface of the micro porous ePTFE sheet. After embossing, the embossed ePTFE sheets are cut into a 25 mm×30 mm rectangular shape for packaging and sterilization by ethylene oxide.

### EXAMPLE 2

### Aging Study

20 pieces of ethylene oxide sterilized textured micro porous ePTFE membranes made in accordance with Example 1 are used in this study. The micro and marco surface textures of the membrane can be examined by microscope, such as a light microscope or scanning electron microscope at magnifications ranging from 10× to 200×. The 25×30 mm rectangular sheets are placed in an oven set at an elevated temperature to speed up the aging of the material. In accordance with Arrhenius Equation, every ten degree Celsius increase in temperature would double the speed of aging. After simulating up to 4 years of aging at room temperature, the stability of the texture pattern were examined and compared with a non-aged sample at 6× magnification. There were no significant differences in micro and macro surface textures between the aged and non-aged control samples.

### EXAMPLE 3

### Clinical Study

Five sterilized, surface textured micro porous ePTFE membranes prepared according to Example 1 were evaluated clinically by practitioners using a flapless, minimally invasive extraction and implant placement combined with guided bone regeneration. The barrier membrane was found readily attached by the surrounding tissue and there were no inflammation or infection due to the use of the barrier membranes. At the completion of the bone regeneration, the membranes were removed using non-traumatic procedures. The result of the study confirms that usefulness of the barrier membranes prepared according to the present invention.

### Reinforced PTFE Biomaterials

Polytetrafluoroethylene (PTFE) and expanded polytetrafluoroethylene (ePTFE) materials are proven biocompatible materials. Articles made by PTFE and ePTFE are used in many medical applications. For example, PTFE and ePTFE membranes are used in guided tissue regeneration or guided bone regeneration for bone grafting applications. Guided bone regeneration (GBR) and guided tissue regeneration (GTR) are dental surgical procedures that utilize barrier membranes to direct the growth of new bone and gingival tissue at sites having insufficient volumes or dimensions of bone or gingival for proper function, esthetics or prosthetic restoration.

Fluorinated ethylene propylene (FEP) is a copolymer of hexafluoropropylene and tetrafluoroethylene. It differs from PTFE resins in that it is melt-processible using conventional injection molding and screw extrusion techniques. It is noted that FEP has a melting point of 260° C. and PTFE decomposes before melting. The melting point of FEP is significantly lower than that of the decomposition temperature of PTFE. ePTFE is made by PTFE in an expansion process, so the thermal and chemical properties are substantially similar to PTFE. Varying grades of FEP films ranging from 1 μm in thickness and up are commercially available. Alternatively, the FEP film may be introduced by spraying a solution of FEP dispersions onto the PTFE or ePTFE membranes before subjecting the layered films under heat and pressure. FEP dispersions are colloid solution containing FEP micro particles suspended in water. The micro particles dispersed onto the PTFE and ePTFE membranes form a FEP film under heat and pressure to bond to the ePTFE membrane.

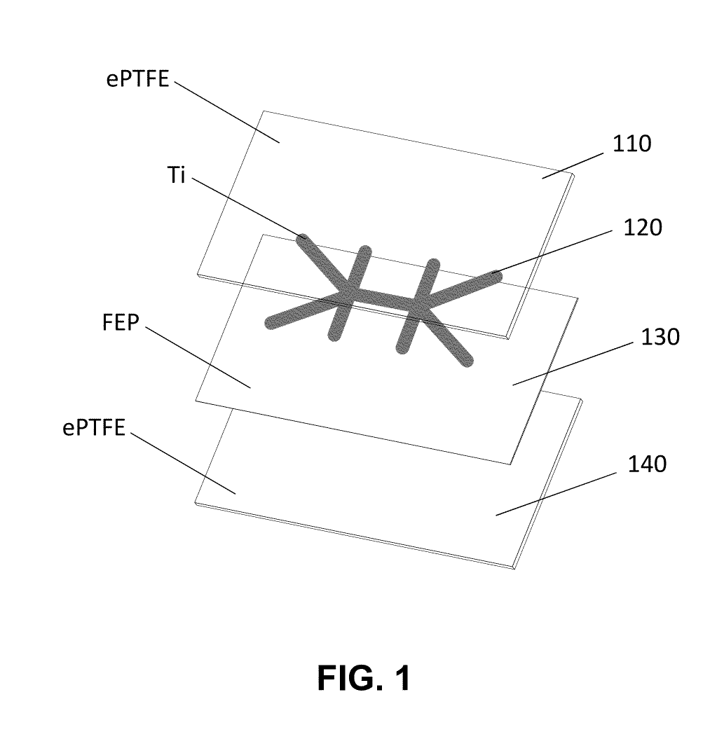

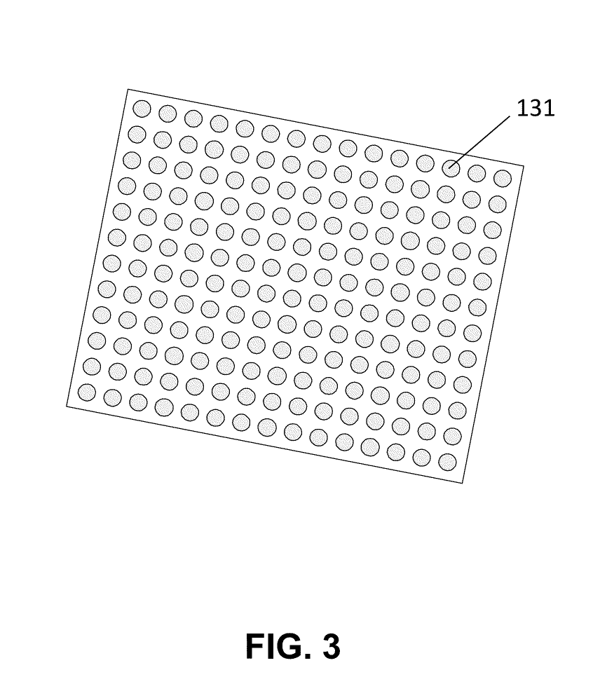

In one aspect, a reinforced biomaterial may include a first layer 110 made by expanded polytetrafluoroethylene polymer (ePTFE), a second layer, which is reinforcing titanium binder 120, a third layer 130 made by fluorinated ethylene propylene polymer (FEP), and a fourth layer 140 made by PTFE, wherein the titanium binder 120 and the FEP layer 130 are sandwiched between the first layer ePTFE 110 and the fourth layer 140, as shown in FIG. 1. In one embodiment, the third layer 130, namely the FEP layer, can be selected from either an FEP film, or a colloidal dispersion of FEP. The thickness of the FEP layer 130 can be ranging from (1) 1 μm to 4 μm; (2) 0.5 μm to 1 μm; and (3) less than or equal to 0.5 μm. In another embodiment, the FEP layer is configured as a mesh with a plurality of holes 131 populated throughout the layer, as shown in FIG. 3. Preferably, the hole size may range from 50 mm² to 100 mm². More preferably, the hole size may range from 10 mm² to 50 mm². Still more preferably, the hole size may range from 4 mm² to 10 mm². More preferably, the hole size can range from 0.01 mm² to 4 mm². The percentage of the open area can be selected from one of the following ranges: (A) from 1% to 20%, (B) from 21% to 40%, (C) from 41% to 60% and (D) greater than 60%.

In a further embodiment, the size of the FEP layer 130 is smaller than the first and optionally the fourth layers (110, 140), namely ePTFE layers. In still a further embodiment, the reinforced biomaterial is used as a membrane for GBR or GTR barrier.

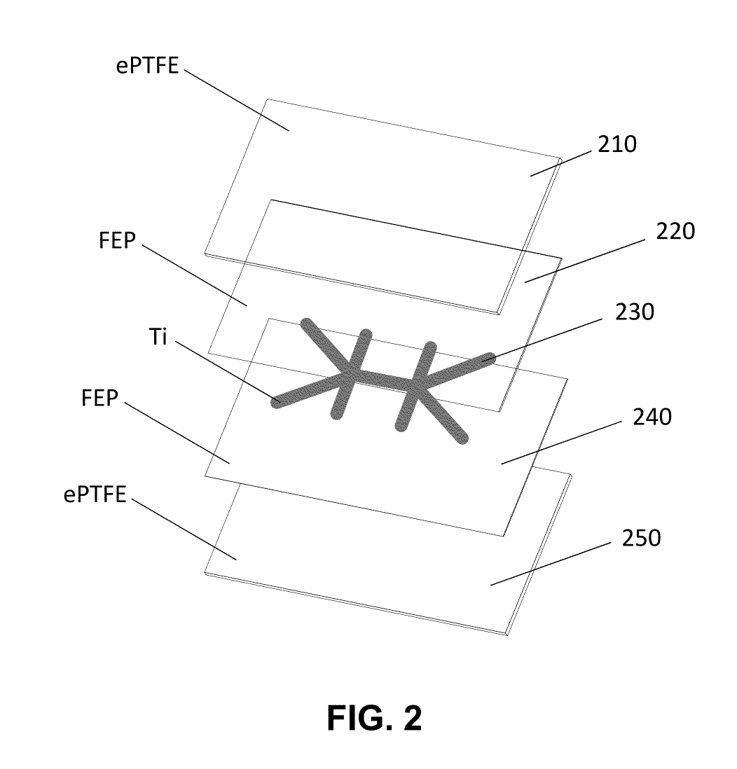

In another aspect, a reinforced biomaterial may include a first layer 210 made by expanded polytetrafluoroethylene polymer (ePTFE), a second layer 220 made by fluorinated ethylene propylene polymer (FEP), third layer made by a reinforcing titanium binder 230, a fourth layer 240 made by FEP, and a fifth 250 layer made by ePTFE, as shown in FIG. 2. In one embodiment, the second and the fourth layers (220, 240), namely the FEP layers, can be selected from either an FEP film, or a colloidal dispersion of FEP. The thickness of the FEP layer can be ranging from (1) 1 μm to 2 μm; (2) 0.5 μm to 1 μm; and (3) less than or equal to 0.5 μm. In another embodiment, the FEP layer is configured as mesh with holes 131 populated throughout the layer, as shown in FIG. 3. Preferably, the hole size may range from 50 mm² to 100 mm². More preferably, the hole size may range from 10 mm² to 50 mm². Still more preferably, the hole size may range from 4 mm² to 10 mm². More preferably, the hole size can range from 0.01 mm² to 4 mm². The percentage of the open area can be selected from one of the following ranges: (A) from 1% to 20%, (B) from 21% to 40%, (C) from 41% to 60% and (D) greater than 60%.

In a further embodiment, the size of the FEP layers (220, 240) is smaller than the ePTFE layers. In a further embodiment, the reinforced biomaterial is used as GBR or GTR barrier membrane.

In another embodiment, the ePTFE layer can be selected from one of the following materials: (1) unsintered, unexpanded polytetrafluoroethylene; and (2) expanded polytetrafluoroethylene. In another embodiment, the thickness of the first and fourth layers can be, respectively, selected from one of the following ranges: (1) 0.6 mm to 1 mm; (2) 0.3 mm to 0.6 mm; (3) 0.1 mm to 0.3 mm; and (4) less than or equal to 0.1 mm. Likewise, the FEP layers, can be selected, respectively, from either an FEP film, or a colloidal dispersion of FEP. The thickness of the FEP layer can be ranging from (1) 1 μm to 2 μm; (2) 0.5 μm to 1 μm; and (3) less than or equal to 0.5 μm. Preferably, the hole size may range from 50 mm² to 100 mm². More preferably, the hole size may range from 10 mm² to 50 mm². Still more preferably, the hole size may range from 4 mm² to 10 mm². More preferably, the hole size can range from 0.01 mm² to 4 mm². The percentage of the open area can be selected from one of the following ranges: (A) from 1% to 20%, (B) from 21% to 40%, (C) from 41% to 60% and (D) greater than 60%. The use of perforated FEP material allows body fluids to diffuse across the multi-layered biomaterial without being impeded by the less permeable FEP layer. Perforation of FEP material also reduces the overall rigidity of the multi-layered material, improving its ability to conform to irregularly shaped wound site. In a further embodiment, the third layer 230 can be selected from one of the following materials: (1) titanium; (2) stainless steel; and (3) other biocompatible metals; and the thickness of the third layer can be selected from one of the following ranges: (1) 4 μm to 6 μm; (2) 2 μm to 4 μm; and (3) less than or equal to 2 μm. The third layer can be shaped in a variety of configuration with an objective to improve the formability of the reinforced multi-layered biomaterial such as disclosed in U.S. Pat. No. 8,556,990. In one embodiment, the biocompatible metal layer is made of a biocompatible metal mesh with holes populated throughout the metal layer. In another embodiment, the biocompatible metal layer has one or more holes suitable for securing a fastener, such as a surgical screw or pin, to the bony defect. In still another embodiment, the biocompatible metal layer has multiple elongated members forming a junction.

In general, the biocompatible membranes of the present invention can be made by laminating the multi-layered components, as described previously, in a mold under heat and pressure. For example, for multi-layered membranes, such as PTFE-FEP-Ti-FEP-ePTFE can be placed in a mold under a predetermined pressure. The mold is then placed in an oven set at a temperature and duration sufficient to cause lamination or bonding of the multi-layered components. After the lamination process is completed, the mold is allowed to cool down to ambient temperature and the membrane is removed from the mold for inspection, trimming, cleaning, testing, and if appropriate, for packaging and sterilization. In general, the lamination should take place at or above the melting temperature of the FEP, and under sufficient pressure and duration to allow bonding of the multi-layered membranes to occur effectively. The pressure used for the bonding process should be greater than ambient pressure, and should be sufficient to hold the multi-layered membranes to intimately contact with each other without gaps in between. The selection of the lamination conditions, including temperature, pressure and duration, depends on the design of the mold and configuration of the final product, such as size, thickness and configurations. The optimal conditions can be determined by experimentation as well known by those with skill of the arts. Typically, the lamination should take place under a pressure that would allow intimate contact between the FEP layer and the layers of Polytetrafluoroethylene (PTFE) material, expanded polytetrafluoroethylene (ePTFE) material and/or the biocompatible metal above and below the FEP layer, at a temperature above the melting point (260° C.) of FEP to allow the FEP to melt into a molten fluid, and at a duration that would allow the molten FEP to fuse into the adjacent material layers. Once these conditions are attained, the mold can be allowed to gradually cool to room temperature. The pressure is then released and the mold is opened to retrieve the laminated multi-layered biomaterial. Typical conditions, including temperature range of 260° C. to 340° C., pressure range of 10 to 100 psi, and time duration of 5 to 30 minutes may be effective to achieve the lamination of multi-layered biomaterials. Conditions outside of the above ranges can also be utilized to accomplish lamination depending upon the specific molding set up and configurations. In one embodiment, the processing conditions are optimized to minimize the effects of material expansion, shrinkage and air entrapment during the lamination. Preferably, the sheet of multi-layered biomaterial or reinforced multi-layered biomaterial has at least one textured surface, and has substantially the needed strength required for the applications in all directions. The sheet of medical barrier of the present invention has a thickness in a range of about 0.01 mm to about 1.00 mm, preferably in the range of about 0.05 mm to about 0.50 mm, and more preferably in the range of about 0.1 mm to about 0.3 mm. The textured surface may include a continuous dotted pattern of holes, and preferably a continuous woven pattern or a continuous pattern of hills and valley formed in the surface of the sheet. If the sheet has only one textured surface, the valley or the indentations have a depth less than the thickness of the sheet and each valley or indentation has a width of less than 0.5 mm, and preferably less than 0.3 mm. The textured patterns are distributed substantially uniform over the surface of the sheet. If the sheet has textured pattern on both surfaces, the valley or the indentations have a depth less than the half the thickness of the sheet. The textured pattern is repeated at less than 500 microns, preferably less than 200 microns and more preferably at less than 100 microns are distributed uniformly over the surface of the sheet.

The surface texture of the multi-layered biomaterial and the reinforced multi-layered biomaterial can be introduced during or after lamination or molding process. In the preferred embodiment, the embossing step is performed by placing a sheet of patterned metal or polymer mesh on top of the unembossed sheet of the multi-layered biomaterial. The patterned metal or polymer sheet material, such as polyethylene or polypropylene, is harder and has more compressive strength than the micro porous ePTFE material. One of the preferred mesh is a fine pore-size titanium mesh manufactured by Unicare Biomedical, Inc. (California). The titanium mesh has a pattern that is embossed into the polymer sheet. The titanium mesh and the ePTFE sheet are passed together between a pair of rollers, which emboss the pattern of the titanium mesh into one or both surface of the ePTFE sheet. After embossing, the embossed multi-layered biomaterial sheet may be cut into smaller sheets of various shape and size for packaging and distribution. Alternatively, the texture pattern may be introduced during the lamination/molding process by laminating/molding the multi-layered biomaterial between two sheets of metal meshes, such as the fine pore size titanium meshes manufactured by Unicare Biomedical, Inc. In such a process, the texture pattern is generated during the lamination or molding process.

In one embodiment, micro pores are generated on the multi-layered biomaterials for GBR or GTR barrier membrane applications. For example, micro pores populated throughout the multi-layered biomaterial and ranging in size from less than 1.0 mm² to less than 0.1 mm² can be created by laser, electron beams or dies. The presence of the micro pores in multi-layered biomaterials enhances the body fluid diffusion across the barrier membrane.

### EXAMPLE 4

### Reinforced Multi-Layered Biomaterial

Delamination of titanium-reinforced membrane are known to happen and often reported by the practitioners who use the membrane. It often occurs in the least desirable timing during administration of the membrane when the membrane is being bent and formed to fit a certain bone defect contour. Delamination of membrane would render the membrane unusable. To date, there have been no reported test methods to screen out membranes that exhibit weak bonding and may delaminate during administration. A good test should allow one to reliably and efficiently screen membranes that are prone to delamination, but not to screen out the well bonded membranes. After many trials, an effective method was established to screen the poorly bonded membranes from the good ones. The test involves grasping the membrane at both ends and stretching the membrane longitudinally at a certain predetermined speed, for example, at a speed selected from a range of 0.5 inch per minute to 10 inches per minute, to extend the length of the membrane to a degree selected from a range from 20% extension to 200% extension. The selection of the stretching speed and the degree of stretching are dependent upon the construct of the membrane and the bonding strength of the membrane. Under such an established test the poorly bonded membrane will delaminate while the well-bonded membranes will maintain the bonding integrity despite significant membrane distortion by stretching. Such tests can be performed by the use of a tensile testing machine, where both ends of the membrane are held by serrated grips, and stretched with a controlled speed to a predetermined length.

Two batches of membranes were prepared in accordance with the method described above. The construct of the membranes is listed in Table 1. The two batches of membranes are prepared under identical processing conditions, including, temperature and pressure used for lamination and the ePTFE layers and titanium metal member incorporated, except that one batch contains two layers of FEP and the other batch without FEP layers. The laminated membranes appear to be identical and could not be detected visually about the bonding strength and tendency for delamination. Six samples from each batch along with three commercial product samples prepared in accordance with U.S. Pat. No. 8,556,990 were tested in accordance with an established method described above. The test data are listed in Table 2 below. The test result demonstrates that multi-layer membranes prepared in accordance with the present invention exhibit superior bonding properties than those from the prior arts.

TABLE 1

| Lot ID | 011314-1 | 011314-1 NoFEP |
|---|---|---|
| Membrane construct | ePTFE/FEP/Ti*/FEP/ | ePTFE/Ti*/ePTFE |
| | ePTFE | |
| Membrane dimensions | 23 × 29 mm | 23 × 29 mm |
| Membrane thickness | 0.010 inches | 0.009 inches |
| Titanium binder thickness | 0.004 inches | 0.004 inches |

*Ti stands for titanium metal member

TABLE 2

| Sample ID | 011314-1 | 011314-1 NoFEP | A047038** |
|---|---|---|---|
| 1 | No delamination | Delamination | Delamination |
| 2 | No delamination | Delamination | Delamination |
| 3 | No delamination | Delamination | Delamination |
| 4 | No delamination | Delamination | — |
| 5 | No delamination | Delamination | — |
| 6 | No delamination | Delamination | — |

**Cytoplast Titanium Reinforced Membrane from Osteogenics Biomedical

### EXAMPLE 5

### Design of Titanium Binder

Applying non-resorbable titanium reinforced membranes made of PTFE, either high density PTFE, expanded PTFE, including textured, or non-textured PTFE membranes for bone grafting is technique sensitive, in particular if the membrane needs to be fixed with bone screw or bone pin to the underlying bone. Fixation of barrier membrane with bone screw or bone pin provides a more stable fixation of bone graft material underneath the membrane, which facilitates bone regeneration. It is known that loose fixation may result in loss of bone graft particles and/or micro movement of bone graft particles, both impede full bone regeneration.

Usually, a multi-layered titanium reinforced membrane is fixed with two bone pins crestally (top of the ridge) and with 3 pins buccally (at the side), although more fixation points may be needed for large size membranes. If there are no holes in the titanium elongated members, bone pins or screws were fastened through the membranes directly. In general the adjacent bone surrounding the bone defect, where the peripheral of the membrane are to be fixated into are uneven. It is known that fixation of the membrane through a titanium binder fixation hole provides a stronger and evener fixation around the fixation point. Once the fixation of membrane is completed, the soft tissue flaps (buccal flap and lingual flap) are lifted up and approximate with each other at the crest to cover the membrane and sutured. The soft tissue flaps are very thin and delicate, and after it is lay on top of the non-resorbable membrane, it is deprived of the blood and nutrients supply from underneath, and have high tendency to become perforate if the membrane are uneven or have sharp points. With appropriate membrane fixation technique to create a smooth contour without sharp corner or edge, maintaining the health of the flap is critical to prevent it from dehiscence, necrosis, sloughing off and/or perforation by the membrane. Membrane that has sharp edge or unsmooth contour tends to perforate the soft tissue. Since the membrane can only be fixed onto solid bone around the edge of the bone defects, selection of the fixation point is important.

Insertion of bone pin or screw is a delicate process and needs to be done carefully with attention to details. Since surgical bone pins and screws are expensive, less fixation points would be preferred to save time and cost. Insertion of bone pin or bone screw tends to generate wrinkles on the membranes, and gaps between the membrane and the bone. Such wrinkles and gaps are undesirable and may cause dehiscence and/or perforation. Thus, there is a need to have a membrane that can reduce dehiscence and perforation after membranes are fixated with bone screw or bone pins, but without affecting the inertness, pliability, malleability and strength of the membrane. This invention provides a Ti-reinforced membrane with design features that will minimize the aforementioned deficiency.

It is difficult to apply bone pin or bone screw because the surgical bone pins and screws used for fixation of membranes are small, ranging from 4 mm to 10 mm in length and 1.2 to 2 mm in thread diameter in general, while the head thereof is about 2.5 to 3 mm in diameter. The use of such a tiny bone pin and screws in a small, tight oral environment requires the use of specially design bone screw/pin fixation kit. The driver tip of the fixation device and the head of the bone pin and screw are designed to engage and to prevent the bone pin from dropping during the process. Even so, dropping of bone pin or bone screw are common during the procedures, partially due to the design of the bone fixation kit, as well as the membrane design. Also, it is due to the fact that the process needs to be carried in a small environment. More seriously, dropping the bone pin or bone screw in the mouth is frustrating to the practitioner, and could be dangerous to the patient, as the patient may accidentally swallow the bone pin or bone screw. Searching and picking up the bone pin or bone screw from the oral cavity is not tedious and re-locking the bone pin or bone screw to the driver tip has to be done carefully to make sure that the bone pin and bone screw are locked to the driver and preventing it from dropping off into the oral cavity again.

In general, the portion of the membrane covering the lingual or buccal side of the defect is not in direct view of the practitioner, so the patient's head or the practitioner's head has to tilt to insert and fasten the bone pin or bone screw. Ideally the bone pin or bone screw should be inserted into the bone at 90 degree angle which gives most depth of penetration and fixation. However, it is difficult to insert the pins or screws at a perfect 90 degree angle. Thus, anything that can smooth or ease the fixation of the bone pin and screw is a great relieve to the practitioner. As discussed above, U.S. Pat. No. 8,556,990 (“the '990 patent”) teaches a multi-layered titanium reinforced membrane with a titanium binder having a pre-drilled hole located at the distal end of the slender elongated member, and the '990 patent is disadvantageous because the pre-drilled hole is very small, and each pre-drilled hole is located at a specific location. The size of the pre-drilled hole is limited by the slenderness of the elongated member, which needs to maintain a narrow width for ease of bending and does not adversely affect the bonding strength of the PTFE layers. If the screw holes are small and are located in specific locations, it is difficult to choose a fixation location. Small holes not only impede the insertion of bone pins and bone screws, but also impede the extraction of the fastener after the bone regeneration has completed. Thus, the present invention is to provide design features that allow bigger holes and elongated slots for fixation and provides stable and smooth fixation point contour.

In summary, in the '990 patent, the size of the titanium binder has to be limited because the bonding strength between the PTFE layers is not very strong so the size of the titanium binder has to be limited to avoid delamination. However, as discussed above, the size of the pre-drilled hole is too small so the practitioners usually have difficult time to secure the screw on the small pre-drilled hole, which may lead to a serious problem during the oral surgical procedure. In the present invention, the FEP layer is introduced between the PTFE layers to significantly increase the bonding strength, so the size of the titanium binder can be greater to provide one or more appendages to replace the pre-drilled holes.

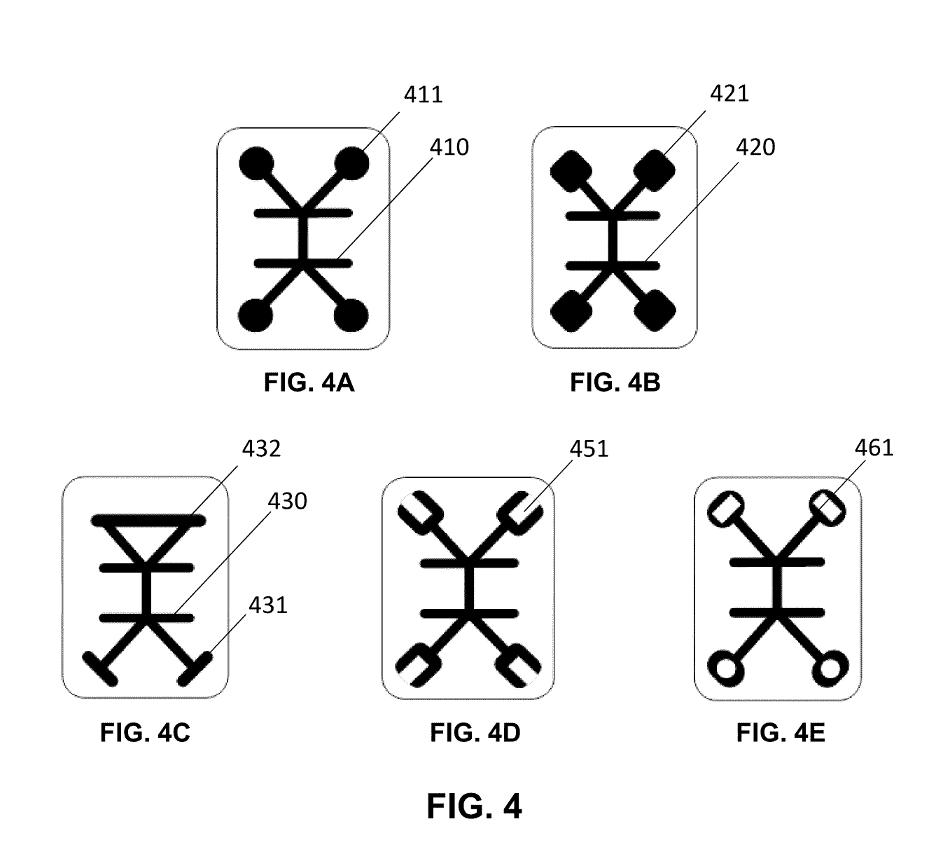

In one embodiment, the reinforcing titanium binder 410 has a plurality of circular appendages 411 at the free distal end of the elongated member spaced apart from the junction, as shown in FIG. 4A. Likewise, the titanium binder 420 has a plurality of rectangular appendage 421 at the free distal end of the elongated members spaced apart from the junction, as shown in FIG. 4B. In another embodiment, the titanium binder 430 has a branch shape appendage 431 at the free distal end of two elongated members spaced apart from the junction, and a connected branch appendage 432 at the distal end of other two now connected elongated members, as shown in FIG. 4C. Such appendages at the distal end of the elongated members of the titanium binder improve the conformance and attachment of the multi-layered membrane to the peripheral contour of bony defect, and significantly reduce the need for the amount of multiple fasteners.

The size and configuration of the appendages at the free distal end of the elongated members are designed to improve the utility of the multi-layered membrane and lower the learning curve for the aspiring practitioners interested in using the multi-layered membranes. The configurations of the appendage can be tailored depending upon the size of the bony defect and dimension of the multi-layered membrane to improve the conforming attachment of the multi-layered membrane without negatively affecting its strength in substantially all directions, inertness, pliability, malleability, resistance to compressive forces, and surface properties of the multi-layered membranes. The overall shape of the binder may be selected to achieve a desired strength, load distribution, barrier support, placement of fasteners, comfort, ease of insertion and/or removal and so on and still allows the multi-layered membranes to be bent, twisted and/or stretched as necessary to obtain the desired shape. The appendage may be configured to have open (441) or enclosed space (451), for example in the form of slot or hole as illustrated in FIGS. 4D and 4E, respectively, to improve the diffusion of body fluid across the membrane, reduce the amount of titanium material used, increase the flexibility of the membrane, have more direct attachment between the fluoropolymers enclosing the titanium binder, and allow the placement of fasteners through the membrane within the appendage. The large void space or elongated slots within the enlarged appendage facilitates the placement of the fastener within relative to the pin hole size located at a slender free distal end of elongated members as disclosed in U.S. Pat. No. 8,556,990. Other shape, size and configuration of the void space within the appendage may be used to facility the utility and/or improving the properties and performance as well.

The reinforcing binder having an appendage located at the free distal end of the joint elongated members can be made of other materials in addition to titanium or surgical grade titanium. For example, the reinforcing titanium binder can be made of any material suitable for medical applications, including implant application that exhibit suitable bendability, compatibility with the enclosing membranes and have a young's modulus or stiffness higher than the enclosing membranes to reinforce the conformance properties of the resulting multi-layered membranes. Such material may be derived from a variety of materials, including polymers, metallic materials, ceramic material, fibrous material or natural polymers, including human or animal tissue, such as collagen, or a composite of the above materials.

Similarly, the multi-layered membranes enclosing the reinforcing binder with an appendage located at the free distal end as described and delineated above can be selected from materials other than PTFE. Any medical grade materials, including implantable material that exhibit flexibility, including flexibility only in the wet state, can be employed to enclose the reinforcing binder, as long as such membrane material can be bonded or attached to the reinforcing binder and exhibit a utility suitable for use a multi-layered membrane for bone grafting applications. Such material may be derived from a variety of materials, including polymers, metallic materials, ceramic material, fibrous material or natural polymers, including plant, human or animal tissue, such as cellulose, collagen, or a composite of the above materials.

Having described the invention by the description and illustrations above, it should be understood that these are exemplary of the invention and are not to be considered as limiting. Accordingly, the invention is not to be considered as limited by the foregoing description, but includes any equivalents.

What is claimed is:

1. A multi-layered membrane, comprising a first layer made by polytetrafluoroethylene polymer (PTFE), a second layer made by fluorinated ethylene propylene polymer (FEP), a third layer made by a reinforcing unit, and a fourth layer made by polytetrafluoroethylene polymer (PTFE), wherein the second and third layers are sandwiched between the first layer and the fourth layer, and the reinforcing unit is configured to span a bone cavity and used for fixation to bone surrounding the bone cavity, wherein the reinforcing unit is configured to form a desired shape prior to placement on the bone defect and maintain the formed shape upon placement.

2. The multi-layered membrane of claim 1, further comprising a fifth layer made by FEP disposed between the third layer and the fourth layer.

3. The multi-layered membrane of claim 1, wherein each of the first layer and the fourth layer is selected from one of the following materials: (1) unsintered, high-density polytetrafluoroethylene; and (2) expanded polytetrafluoroethylene.

4. The multi-layered membrane of claim 2, wherein the second and the fifth layer, are selected from an FEP film, or a colloidal dispersion of FEP.

5. The multi-layered membrane of claim 2, wherein thickness of the each FEP layer is selected from one of the following ranges: (1) 0.05 mm; (2) 0.025 mm; and (3) less than 0.025 mm.

6. The multi-layered membrane of claim 1, wherein the second layer is configured as a mesh with a plurality of holes populated throughout the second layer, allowing the PTFE layers attached at one or more points to each other.

7. The multi-layered membrane of claim 6, wherein the percentage of the mesh open area is selected from one of the following ranges: (1) from 1% to 20%; (2) from 21% to 40%; (3) from 41% to 60%; and (4) from 60% to 80%.

8. The multi-layered membrane of claim 2, wherein the two FEP layers have open structures allowing the external polytetraflouroethylene layers attached at one or more points to each other, wherein the percentage of open area of each FEP layer is selected from one of the following ranges: (A) less than 10%, (B) 10% to 30%, (C) 30% to 50% and (D) 50% to 80%.

9. The multi-layered membrane of claim 1, wherein size of the FEP layer is smaller than PTFE layers.

10. The multi-layered membrane of claim 1, wherein the reinforcing unit is comprised of a material with young's modulus greater than any other layers, and is selected from one of the following materials: (A) metal, (B) synthetic polymer, (C) natural polymer (D) fibrous material, (F) ceramic, and (G) a composite material including one or more of the above mentioned materials.

11. The multi-layered membrane of claim 1, wherein thickness of the reinforcing unit is selected from one of the following ranges: (1) 0.1 mm to 0.2 mm; (2) 0.05 mm to 0.1 mm; and (3) less than or equal to 0.05 mm.

12. The multi-layered membrane of claim 1, wherein the reinforcing unit is made with a configuration selected from one of the following: (1) mesh with holes populated throughout; (2) having multiple elongated members forming a junction or junctions, and the elongated members have a free distal end that extends away from the junction; (3) having multiple elongated members forming a junction or junctions, and the elongated members have a free distal end that extends away from the junction; wherein at least one of the elongated member has a hole formed in the free distal end spaced apart from the junction, and (4) having multiple elongated members forming a junction or junctions, and the elongate members have a free distal end that extends away from the junction; wherein at least one of the elongated member has an enlarged appendage formed in the free distal end spaced apart from the junction.

13. The multi-layered membrane of claim 12, wherein the appendage at the free distal end of the elongated member of the reinforcing unit is formed in one of the following selected shapes: (A) circular, (B) rectangular, (C) elliptical, (D) branch, and (D) anyone of the above with open structure within the appendage.

14. The multi-layered membrane of claim 1, wherein the composite multi-layered material is used as a medical barrier membrane for guided tissue regeneration.

15. The multi-layered membrane of claim 1, wherein at least one PTFE layer having a textured surface.

16. A multi-layered membrane comprising a reinforcing unit configured to span a bone cavity and used for fixation to bone surrounding the bone cavity, and said reinforcing unit sandwiched between two or more layers of polymeric membrane, wherein said reinforcing unit has multiple elongated members forming a junction or junctions, and the elongated members have a free distal end that extends away from the junction, and at least one elongated member has an enlarged appendage formed in the free distal end spaced apart from the junction.

17. The multi-layered membrane of claim 16, wherein the size, configuration and location of the appendages relative to the junction vary depends upon the size of the multi-layered membrane, and the enlarged appendage improves the conforming attachment of the multi-layered membrane to the contour of the bony defect, and reduce the need for multiple fasteners to secure the membrane to the bone.

18. The multi-layered membrane of claim 16, wherein the appendages of the reinforcing unit are made in one of the following shapes: (A) circular, (B) rectangular, (C) elliptical, (D) branch and (E) irregular shape.

19. The multi-layered membrane of claim 18, wherein the appendages of the reinforcing unit further comprise an open or enclosed void area, and the open area allows the enclosing layers to contact each other, and the void area is shaped in one of the following configurations: (A) circular, (B) rectangular, (C) elliptical, (D) slot, and (E) irregular.

20. The multi-layered membrane of claim 16, wherein a portion of a top layer of the polymeric membrane is attached to a portion of a bottom layer of the polymeric membrane.

21. The multi-layered membrane of claim 20, wherein the layers of polymeric membranes are comprised of one or more of the following materials: (A) synthetic polymers, (B) natural polymers, (D) resorbable polymers, (E) hydrophilic polymers, (F) cellulose, (G) collagen, (H) chitosan, and (I) a composite material including one or more of the above mentioned materials, said the thickness of the multi layer membrane is less than 0.5 mm

22. The multi-layered membrane of claim 20, wherein the layers of polymeric membrane comprises of fluoropolymers selected from the one or more of the following materials: (A) polytetrafluoroethylene, (B) fluorinated ethylene propylene polymer, (C) perfluoroalkoxy polymer, (D) polyethylenetetrafluoroethylene, (E) polyethylenechlorotrifluoroethylene, (F) perfluoropolyether, (G) polyvinylidene fluoride, (H) polyvinylfluoride, (I) expanded polytetraflouroethylene, (J) unsintered polytetraflouroethylene has a density of about 1.2 gm/cc to about 2.3 gm/cc, and (K) a composite of above mentioned polymer membranes.

24. The multi-layered membrane of claim 16, wherein at least one surface of the polymeric membrane layer is textured.

25. The multi-layered membrane of claim 16, wherein the reinforcing unit comprises of material selected from one of the following: (A) metal, (B) synthetic polymer, (C) natural polymer, (D) fibrous material, (E) ceramic, and (F) a composite of the abovementioned material.

26. The multi-layered membrane of claim 16, wherein the reinforcing unit comprises of titanium foil.
